(19) United States
(12) Patent Application Publication
(10) Pub. No.: US 2009/0062809 A1
(43) Pub. Date: Mar. 5, 2009

(54) Cartilage bore depth gauge

(75) Inventor: Anton J. Steiner, Wharton, NJ (US)

Correspondence Address: JOHN S. HALE;GIPPLE & HALE, 6665-A OLD DOMINION DRIVE, MCLEAN, VA, 22101, US

(73) Assignee: Musculoskeletal Transplant Foundation

(21) Appl. No.: 11/896,077

(22) Filed: Aug. 29, 2007

(51) Int. Cl. A61B 17/58; B65D 83/00

(52) U.S. Cl. 606/102; 206/363

(57) ABSTRACT

The invention is directed toward a depth gauge instrument for simultaneously taking a number of height measurements around the surface of the area surrounding a bore from the base of the bore. The instrument has an individual cylindrical base member which has a diameter the same as the bore allowing the same to be inserted in the bore and a plurality of marker members slideably mounted in dovetail shaped grooves cut longitudinally along the outer surface of the cylindrical base member. The base member is provided with measurement indicia on its outer surface which correspond to the foot of each marker members to show the specific distance that the marker member is from the base of the bore.

### RELATED APPLICATIONS

There is no related application.

### STATEMENT REGARDING FEDERALLY SPONSORED RESEARCH OR DEVELOPMENT

Not applicable.

### REFERENCE TO SEQUENCE LISTING, A TABLE OR A COMPUTER PROGRAM LISTING COMPACT DISC APPENDIX

None.

### BACKGROUND OF THE INVENTION

1. Field of Invention

The present invention is generally directed toward a device for measuring a bore depth and more particularly, a device for providing multiple measurements of the cartilage surface area surrounding a cartilage defect bore in relation to the depth of the bore.

2. Background of the Invention

In arthroscopic surgical procedures for alleviating a cartilage defect, it is typical to remove the defect by using a bit to remove the cartilage area and underlying subchondral bone leaving a blind bore in the defect area.

Because the surface cartilage area is typically rounded, the height of the cartilage surface from the bottom of the bore taken around the periphery of the bore differs. Even though an implant with the correct diameter can be easily determined, it is difficult for the cartilage cap or top surface of the implant to align with the cartilage surface area surrounding the bore.

At present there is no known method of determining precisely how much bone or implant material should be removed from the implant plug, nor of gauging how the plug should be trimmed for correct cartilage surface alignment with the surrounding cartilage area. These implant procedures are performed with an interference fit with a cylindrical implant plug being driven into the defect bore. Once the implant plug is seated in the bore, it is difficult to remove due to the tight interference fit with the bore walls. Consequently, a surgeon may remove an insufficient amount of bone from the implant, creating an inadequate fit as the implant is raised above the cartilage surface or remove to much bone from the implant causing the implant upper surface to be lower than the cartilage surface, leaving the patient with an implant top surface which does not match the contour of the surrounding cartilage area. Since the cartilage surface is a bearing surface, such raised or unequal edges of the implant can cause wear on the replacement site or present a friction area for the overlying joint surface with its adjacent hyaline cartilage surface.

During drilling or removal of the cartilage defect area, the surgeon is typically capable of drilling a bore of predetermined depth and diameter. Because the simple act of drilling does not provide an exact measurement of the depth of the bore itself, a depth gauge is commonly employed for directly measuring the depth of the hole from the top surface to make sure that the implant plug is trimmed correctly. However, this is a single depth measurement and does not take into consideration the differences of height taken from different positions of the cartilage surface around the periphery of the bore.

Currently, many devices are known and utilized for measuring the depth of a hole or bore in a portion of a bone. Generally speaking, these devices utilize a central probe member having a barb at a distal end, and a sleeve or channel member.

Proper reading of the markings on such device requires a surgeon's eyes to be properly aligned with the markings which may be obscured with blood and/or bone and cartilage fragments. Generally, the measurement of reading the markings requires the surgeon to view the gauge from a lateral point of view so that the view of the depth gauge marker aligned with the graduated markings is proper and not distorted by the surgeon's elevated, standing perspective. Therefore, it is often necessary for the surgeon to bend over while using these gauges to view a reading which only gives the depth of the bore at that particular measurement point.

U.S. Pat. No. 6,042,584 issued Mar. 28, 2000 shows a gauge for measuring bone thickness which includes an elongated pin for insertion into a bore through a bone section. The pin has a length sufficient to span the entire bone section with a distal tip protruding therefrom. The pin further has a length measurement indicator disposed along an outer surface, such as a series of colored bands or indicia, the visualization of which communicates bone thickness. The gauge also has a head at a proximal end of the pin, which is dimensioned to prevent its movement into the bone section's bore.

U.S. Pat. No. 7,165,336 issued Jan. 23, 2007 shows a depth gauge for measuring the depth of a hole in a bone having a digital readout for providing visual measurement to a surgeon. The depth gauge has a probe with a tip inserted into and positioned proximate to the depth of the hole, and has a reference member adjustably positioned relative the probe and against the bone proximate the hole. A measuring device is provided with the depth gauge for measuring the relative distance between the tip and the reference member, and the measuring device has a large, digital display for providing the relative distance.

U.S. Pat. No. 5,013,318 shows a medical instrument for measuring depth of a fastener hole cut in bone. The depth gauge has a scale tube which fits into a fastener hole in the bone. The zero index of scale tube expands exclusively in radial dimension to a diameter greater than the hole diameter, affording a positive stop for the zero index, thus insuring alignment of scale zero index with edge of hole in distal bone wall. A sliding scale marker is frictionally mounted to scale tube, affording a record of the measurement.

U.S. Pat. No. 6,836,969 issued Jan. 4, 2005 shows a hand held device for making measured markings. The device comprises a body having a bottom and a side surface with an abutting surface connected to the body having a flat face perpendicular to the body to slidably butt against an edge of a work piece. A plurality of spaced guiding wells adjacent the side surface may receive a marking instrument point. The guiding wells simultaneously provide measurement and guidance for the marking instrument while the abutting surface is slid along the edge of a work piece. A telescoping member having a flat top edge provides adjustable guidance for marking instrument as well. Reference markings on the telescoping member mark the distance from the edge of the telescoping member to the butting surface.

None of the prior art devices solve the problem of a non planar surface surrounding a bore cut to remove a cartilage defect in providing multiple trim measurements for an implant for the bore.

### SUMMARY OF THE INVENTION

The present invention is directed to a bore depth gauge instrument which measures the relative height of different areas located around a bore taken from the bottom of the bore. The device has a cylindrical base member which has a slightly smaller diameter as the bore being measured, with concentric measurement indicia placed around the outer surface of the cylindrical base member. A plurality of dove tail shaped grooves are cut longitudinally in the outside surface of the base member which are parallel to each other and equidistantly spaced to each other. A plastic depth marker having a correspondingly shaped dove tail slide section shaped to fit in each groove is slideably mounted in each groove and as the base member is inserted into the bore, the top surface of the cartilage around the bore engages the foot of the markers causing them to remain in a stationary position with respect to the top surface while sliding along the respective groove as the cylindrical base member is pushed down into the bore by an integral smaller diameter stem. The stem extends away form the cylindrical base along the center axis of the base and both the base and the stem define a central through going bore running along the central axis of the device. When the planar bottom surface of the base member hits the floor of the bore, the device is removed and each marker remains in position on the base member giving a distance reading from the bottom of the bore to a particular surrounding cartilage surface point.

It is an object of the invention to provide a bore gauge instrument which enables a surgeon to quickly, precisely and conveniently measure the depth of bore and determine the height of surrounding area surface points of the bore.

It is another object of the invention to provide a bore gauge instrument with multiple measuring elements to contact and measure the height of the distal bone wall to the surrounding surface.

It is still another object of the invention to provide a bone gauge instrument with which a surgeon can access any reasonably desired location in a human body.

It is yet another object of the invention to provide a bone gauge instrument which is sized for the bore diameter to be measured and which is lightweight so that a surgeon can use it with ease.

It is another object of the invention to provide a bore gauge instrument which is used alone, without Xray, or additional equipment to determine surface measurements around a bore opening.

These and other objects, advantages, and novel features of the present invention will become apparent when considered with the teachings contained in the detailed disclosure along with the accompanying drawings.

### BRIEF DESCRIPTION OF THE DRAWINGS

FIG. 1 is a perspective view of the inventive depth gauge instrument.

FIG. 2 is an enlarged cross sectional view of the depth gauge instrument of FIG. 1 inserted into a cartilage defect bore;

FIG. 3 is a perspective view of the depth gauge instrument removed from the defect bore of FIG. 2;

FIG. 4 is a side elevation view of the depth gauge instrument of FIG. 1 with the slideable marker members removed;

FIG. 5 is a top plan view of the depth gauge of FIG. 4;

FIG. 6 is an enlarged side elevation view of the marker member;

FIG. 7 is a rear elevation view of the marker member of FIG. 6;

FIG. 8 is a front elevation view of the marker member of FIG. 6; and

FIG. 9 is an end elevation view of the marker member of FIG. 6.

### DESCRIPTION OF THE INVENTION

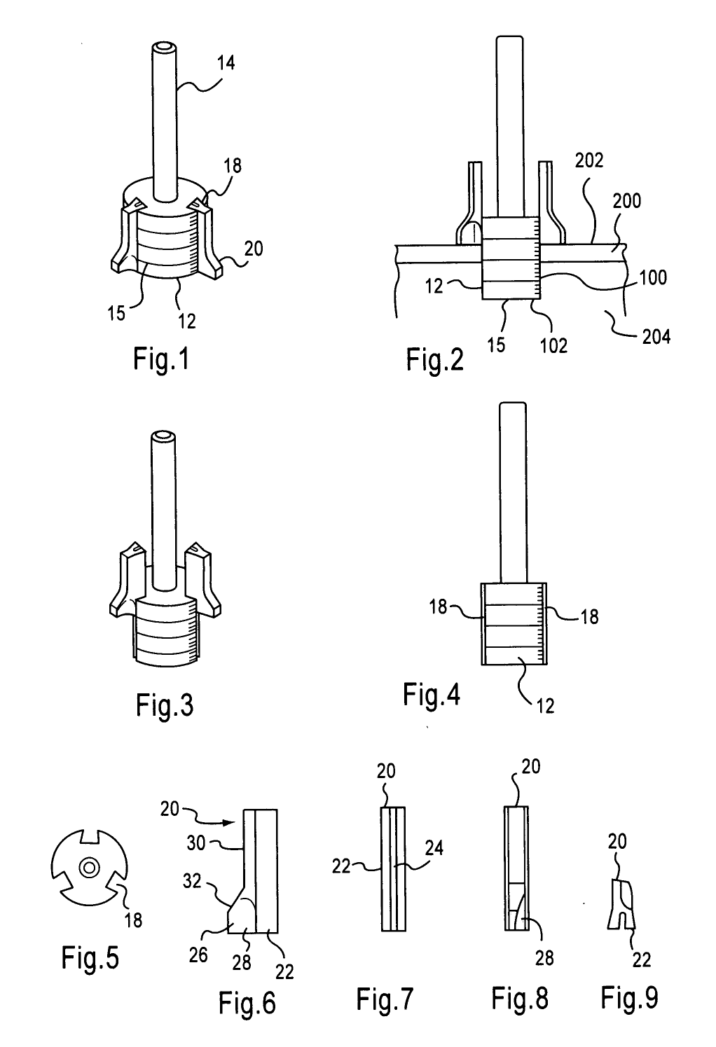

The present invention is directed towards an instrument for measuring the heights of surface areas surrounding a bore from the floor of the bore. The best mode and preferred embodiment of the invention is shown in FIGS. 1-9. The depth measuring instrument 10 is preferably constructed of surgical stainless steel. The instrument 10 has a cylindrical base member 12 having depth measurement indicia 13 marked around its circumference in millimeter increments and has an integral stem 14 extending away from the central axis of the base member 12. The stem 14 and base member 12 define a through going axial bore 16 as seen in FIG. 5 which allows the transport of blood and fluids from the bottom 102 of the cartilage defect bore 100 allowing the planar end surface 15 of the base member 12 to be seated on the bottom 102 of the bore 100. If desired, the upper section of the stem bore 16 can define a threaded portion to receive an extender member or other device. The base member 12 has a plurality of equidistant dove tail shaped grooves 18 cut longitudinally in its outer surface which hold marker members 20 for slideable movement therein. The grooves 18 are preferably located at 120° intervals around the base member 12. The marker members 20 are preferably made of plastic and have a flared dove tail shaped base 22 which fit in grooves 18, the base 22 defining a centrally positioned channel 24 which runs the length of the base 22 allowing the base to easily slide along groove 18. A marker foot 26 extends from the base and indicates the surface area distance from the bottom 102 of bore 100 to the top surface 202 of the cartilage layer 200. The marker foot 26 has an angled portion 28 leading to a flat top outer surface 30 and also defines a downwardly angled section 32.

In operation, a cylindrical bore 100 of predetermined diameter is drilled in a patient to remove a cartilage defect area in the cartilage 200. The bore 100 is cut through the cartilage area 200 and into the subchondral bone 204. It will be appreciated that the top cartilage surface 202 of the cartilage area 200 is rounded and thus different areas around the bore periphery will have varying heights from the bore floor or bottom 102.

The depth measuring instrument 10 having a base member 12 with a diameter slightly smaller than the bore 100 is placed in the bore 100 and the surgeon pushes the instrument 10 via stem 14 until the base member 12 end planar surface 15 is seated on the bore bottom floor 102. The top cartilage surface 202 of the surrounding cartilage area 200 around the bore 100 engages the feet 26 of the marker members 20 causing the marker numbers to slide in their respective grooves 18 until the depth measuring instrument is seated in bore 100. The individual marker members 20 thus show the surrounding surface height from the base of the bore 100 at three separate 120° intervals around the bore 100. The surgeon then reads the height differences around the bore circumference and shapes the implant accordingly so that it has a top surface that will be flush with the surrounding top cartilage surface 202. It is envisioned that a kit having plurality of different diameter size instruments 10 be used so that the surgeon can pick the approximate diameter instrument for the diameter of the bore used to excise the defect area. Base member diameter sizes of the instruments for the kit are 10, 14, 18, 20, 22, 25, 30 and 35 mm in size.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What I claim is:

1. An instrument for measuring the distance of the surface area surrounding a bore from the base of the bore comprising: an individual base member insertable in the bore and a plurality of marker members slideably mounted to said base member, said base member having measurement indicia on its outer surface.

2. An instrument as claimed in claim 1 wherein said base member defines a plurality of equally spaced longitudinal grooves in its outer surface with said marker members being slideably mounted in said grooves.

3. An instrument as claimed in claim 2 wherein said grooves are located at 120° intervals around said outer surface.

4. An instrument as claimed in claim 2 wherein each of said marker members has a base which is of the same configuration as said grooves.

5. An instrument as claimed in claim 4 wherein said marker member base defines a longitudinal channel therein.

6. An instrument as claimed in claim 4 wherein each marker member has a foot section extending from one end of said marker member base.

7. An instrument as claimed in claim 2 wherein said grooves have a dove tail shaped configuration.

8. An instrument as claimed in claim 1 wherein a stem member is integrally formed with said base member and extends from said base member

9. An instrument for measuring the distance of multiple points of a surface area surrounding a bore from the base of the bore comprising: a cylindrical base member insertable in the bore, a stem member mounted to said base member and extending from said base member, said base member defining a plurality of longitudinal grooves in its outer surface and a plurality of marker members slideably mounted in said spaced grooves, said base member having measurement indicia defined on its outer surface.

10. An instrument as claimed in claim 9 wherein said grooves are located at 120° intervals around said outer surface.

11. An instrument as claimed in claim 9 wherein each of said marker members has a base which is of the same configuration as said grooves.

12. An instrument as claimed in claim 9 wherein said marker member base defines a longitudinal channel therein.

13. An instrument as claimed in claim 11 wherein each of said marker members has a foot section extending from one end of said marker member base.

14. An instrument as claimed in claim 9 wherein said grooves have a dove tail shaped configuration.

15. An instrument for measuring the distance of multiple points of a surface area surrounding a bore from the base of the bore comprising: a cylindrical base member insertable in the bore, a stem member extending from said base member, said base member defining a plurality of equally spaced longitudinal grooves in its outer surface having a dove tail shape and a plurality of marker members slideably mounted in said spaced grooves, each of said marker members having a base with a dovetail shape which fits in a groove, said base member having measurement indicia on its outer surface.

16. An instrument as claimed in claim 15 wherein each of the said marker members has a base defining a longitudinal channel therein.

17. An instrument as claimed in claim 16 wherein each of said marker members has a foot extending from one end of said marker member base.

18. An instrument as claimed in claim 17 wherein said foot has an angled section.

19. A kit comprising a container with a plurality of different diameter instruments for measuring the distance of the surface area surrounding a bore from the base of the bore, each instrument comprising: a cylindrical base member for insertion in a bore, said base member defining a plurality of longitudinal dovetail shaped groove, a plurality of marker members slideably mounted in said grooves, said base member being provided measurement indicia on its outer surface.

20. An kit as claimed in claim 19 wherein said different diameter instruments comprise at least two instruments taken from a group of instruments having diameters of 10, 15, 18, 20, 22, 25, 30, and 35 mm in size.
